(19) United States
(12) Patent Application Publication
(10) Pub. No.: US 2026/0022087 A1
(43) Pub. Date: Jan. 22, 2026

(54) PROCESS DEVICE USING IPA AND METHOD THEREOF

(71) Applicant: Shanghai Huali Integrated Circuit Corporation, Shanghai (CN)

(72) Inventor: Chaofei ZHANG, Shanghai (CN)

(73) Assignee: Shanghai Huali Integrated Circuit Corporation, Shanghai (CN)

(21) Appl. No.: 19/087,790

(22) Filed: Mar. 24, 2025

(30) Foreign Application Priority Data
Jul. 19, 2024 (CN) 202410977635.0

(51) Int. Cl. C07C 29/90

(52) U.S. Cl. CPC C07C 29/90

(57) ABSTRACT

The present disclosure discloses a process device using IPA, which comprises: a process tank for storing IPA. The process tank comprises a first inlet and a second inlet. The first inlet is connected to an IPA supply through a first pipeline. The second inlet is connected to a shielding gas supply through a second pipeline; in the process tank, the shielding gas provides an oxygen-free atmosphere, thereby prevents the IPA from being oxidized. The present disclosure further discloses a process method using IPA. The present disclosure can prevent IPA from being oxidized, thereby can prevent residual impurities formed by IPA oxidation.

### CROSS-REFERENCE TO RELATED APPLICATIONS

This application claims priority to Chinese patent application No. 202410977635.0, filed on Jul. 19, 2024, the disclosure of which is incorporated herein by reference in its entirety.

### TECHNICAL FIELD

The present disclosure relates to the field of semiconductor integrated circuit manufacturing, in particular to a process device using isopropanol (IPA). The present disclosure further relates to a process method using IPA.

### BACKGROUND

In advanced technology nodes, IPA drying is widely used in the industry for a drying step after cleaning, and hot IPA can further improve the wettability of chemicals. Therefore, a high-temperature IPA process at 70 degrees is introduced for some multi-layer process layers with a depth-width ratio as high as 10:1. Owing to IPA's inherent characteristic of easy oxidation at high temperatures, a wide range of residues such as acetone will be produced in the actual use of IPA, affecting the product yield.

In addition, owing to the limitations of the existing chemical preparation processes, trace metal ions remain in IPA. At room temperature, IPA is prone to slow oxidation to generate organic impurities such as acetone, and the degree of oxidation becomes more serious with the increase of temperature, resulting in a large quantity of residues that can't be filtered. These residues are usually organic impurities, and cause defects when they are mapped to wafers, for example, a large number of particles in size greater than 60 nm will be formed, ultimately affecting final product yield.

### BRIEF SUMMARY

The technical problem to be solved by the present disclosure is to provide a process device using IPA, which can prevent IPA from being oxidized, thereby can prevent impurity residues resulted from IPA oxidation. To that end, the present disclosure further provides a process method using IPA.

In order to solve the above technical problem, the process device using IPA provided by the present disclosure comprises:

a process tank for storing IPA.

The process tank comprises a first inlet and a second inlet.

The first inlet is connected to an IPA supply through a first pipeline, and the IPA supply is used to supply the IPA into the process tank.

The second inlet is connected to a shielding gas supply through a second pipeline, and the shielding gas supply is used to introduce a shielding gas into the process tank; in the process tank, the shielding gas provides an oxygen-free atmosphere, thereby prevents the IPA from being oxidized.

As a further improvement, the shielding gas at least comprises one or more inert gases or nitrogen, which is used to realize the oxygen-free atmosphere.

As a further improvement, the shielding gas at least comprises one or more reducing gases, which are used to reduce IPA oxides in the process tank.

As a further improvement, the reducing gas comprises hydrogen.

As a further improvement, the shielding gas employs a mixture of oxygen and hydrogen.

As a further improvement, the IPA oxide comprises acetone.

As a further improvement, the process tank comprises a first outlet, a second outlet and a third outlet.

The first outlet is connected to an IPA drain through a third pipeline.

The second outlet is connected to a shielding gas drain through a fourth pipeline.

The third outlet is connected to a process tower through a fifth pipeline, and the process tower is used to deliver the IPA to a process position and realize an IPA treatment process.

As a further improvement, the IPA treatment process comprises an IPA drying process.

As a further improvement, a heater is arranged on the fifth pipeline for heating the IPA flowing therethrough to a temperature required by the IPA treatment process.

As a further improvement, the maximum temperature in the IPA drying process is equal to or above 70 degrees.

As a further improvement, downstream of the heater, the fifth pipeline is connected to the third inlet of the process tank through a sixth pipeline for refluxing the IPA into the process tank.

In order to solve the above technical problem, the process method using IPA provided by the present disclosure comprises the following steps:
storing IPA in a process tank;
introducing a shielding gas into the process tank, which provides an oxygen-free atmosphere in the process tank, thereby prevents the IPA from being oxidized; and
delivering the IPA to a process position and realizing an IPA treatment process.

As a further improvement, the shielding gas at least comprises one or more inert gases or nitrogen, which is used to realize the oxygen-free atmosphere.

As a further improvement, the shielding gas at least comprises one or more reducing gases, which are used to reduce IPA oxides in the process tank.

As a further improvement, the reducing gas comprises hydrogen.

As a further improvement, the shielding gas employs a mixture of oxygen and hydrogen.

As a further improvement, the IPA oxide comprises acetone.

As a further improvement, the IPA treatment process comprises an IPA drying process.

As a further improvement, the IPA further comprises heating the IPA before the IPA is delivered to the process position.

As a further improvement, the maximum temperature in the IPA drying process is equal to or above 70 degrees.

According to the present disclosure, by introducing a shielding gas into the process tank for storing IPA, the IPA in the process tank can be fully in an oxygen-free atmosphere, so that IPA oxidation can be prevented, for example, IPA oxidation can be prevented in the IPA treatment process, especially, IPA oxidation caused by high temperature in the IPA treatment process can be prevented, so that impurity residues resulted from IPA oxidation can be prevented, and pollution of the particles of the object of the IPA treatment process, such as wafer, caused by impurity residues can be reduced or eliminated.

The shielding gas in the present disclosure may further contain a reducing gas, which can reduce IPA oxides already formed in IPA, such as a small amount of IPA oxides formed in the IPA transport chain, thereby further eliminate the adverse effects caused by IPA oxidation. In that way, the present disclosure can solve the technical problems caused by slow oxidation in the transportation process and continuous oxidation of IPA during end use at a high temperature, i.e., the IPA treatment process, and finally can better reduce impurity residues and corresponding particle pollution caused by IPA oxidation.

### BRIEF DESCRIPTION OF THE DRAWINGS

The present disclosure will be further described below in detail in specific embodiments with reference to the accompanying drawings:

FIG. 1 is a structural diagram of the process device using IPA in an embodiment of the present disclosure.

### DETAILED DESCRIPTION OF THE DISCLOSURE

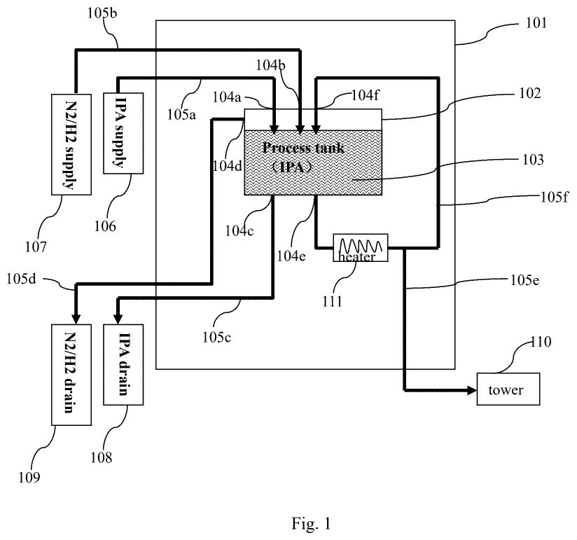

Please refer to FIG. 1, which is a structural diagram of the process device using IPA in an embodiment of the present disclosure; the process device using IPA in the embodiment of the present disclosure comprises:
a process tank 102 for storing IPA; in FIG. 1, the process tank 102 is illustrated, and the area indicated by the reference numeral 103 is an IPA liquid area.

The process tank 102 comprises a first inlet 104a and a second inlet 104b.

The first inlet 104a is connected to an IPA supply 106 through a first pipeline 105a, and the IPA supply 106 is used to supply the IPA into the process tank 102.

The second inlet 104b is connected to a shielding gas supply 107 through a second pipeline 105b, and the shielding gas supply 107 is used to introduce a shielding gas into the process tank 102; in the process tank 102, the shielding gas provides an oxygen-free atmosphere, thereby prevents the IPA from being oxidized.

In an embodiment of the present disclosure, the shielding gas at least comprises one or more inert gases or nitrogen, which is used to realize the oxygen-free atmosphere.

The shielding gas at least comprises one or more reducing gases, which are used to reduce IPA oxides in the process tank 102. The IPA oxide comprises acetone.

In some preferred embodiments, the reducing gas comprises hydrogen.

The shielding gas employs a mixture of oxygen and hydrogen. In FIG. 1, N2/H2 represents a mixture of oxygen and hydrogen. The shielding gas supply 107 is also represented by an N2/H2 supply, and the IPA supply 106 is also represented by an IPA supply.

In an embodiment of the present disclosure, the process tank 102 comprises a first outlet 104c, a second outlet 104d and a third outlet 104e.

The first outlet 104c is connected to an IPA drain 108 through a third pipeline 105c.

The second outlet 104d is connected to a shielding gas drain 109 through a fourth pipeline 105d.

In FIG. 1, the shielding gas drain 109 is also represented by a N2/H2 drain, and the IPA drain 108 is also represented by an IPA drain.

The third outlet 104e is connected to a process tower 110 through a fifth pipeline 105e, and the process tower 110 is used to deliver the IPA to a process position and realize an IPA treatment process. In FIG. 1, the process tower 110 is also represented by a tower.

The IPA treatment process comprises an IPA drying process. In some embodiments, the maximum temperature in the IPA drying process is equal to or above 70 degrees.

A heater 111 is arranged on the fifth pipeline 105e for heating the IPA flowing therethrough to a temperature required by the IPA treatment process.

Downstream of the heater 111, the fifth pipeline 105e is connected to the third inlet 104f of the process tank 102 through a sixth pipeline 105f for refluxing the IPA into the process tank 102.

As shown in FIG. 1, all of the first inlet 104a, the second inlet 104b and the third inlet 104f are located at the top of the process tank 102, which is beneficial for inflow of the shielding gas and the IPA; the first outlet 104c and the third outlet 104e are located at the bottom of the process tank 102, which is beneficial for IPA outflow. The second outlet 104d is located at the top of a side of the process tank 102, and an oxygen-free atmosphere consisting of the shielding gas is formed at the top of the IPA liquid area indicated by the reference numeral 103; the second outlet 104d is located at the top of the side of the process tank 102, which is beneficial for the flow of the shielding gas and thereby provides a satisfactory oxygen-free atmosphere.

In an embodiment of the present disclosure, the process device using IPA is arranged on a wet etching machine 101. The wet etching machine 101 is used to implement wet etching or wet cleaning of wafers, and different etching solutions or cleaning solutions can be selected as required according to the materials to be etched or the objects to be cleaned. After wet etching or wet cleaning is completed, the IPA drying process is used to dry the surface of the wafer.

According to the embodiments of the present disclosure, by introducing a shielding gas into the process tank 102 for storing IPA, the IPA in the process tank 102 can be fully in an oxygen-free atmosphere, so that IPA oxidation can be prevented, for example, IPA oxidation can be prevented in the IPA treatment process, especially, IPA oxidation caused by high temperature in the IPA treatment process can be prevented, so that impurity residues resulted from IPA oxidation can be prevented, and pollution of the particles of the object of the IPA treatment process, such as wafer, caused by impurity residues, usually organic impurity residues, can be reduced or eliminated.

The shielding gas in the embodiments of the present disclosure may further contain a reducing gas, which can reduce IPA oxides already formed in IPA, such as a small amount of IPA oxides formed in the IPA transport chain, thereby further eliminate the adverse effects caused by IPA oxidation. In that way, the embodiments of the present disclosure can solve the technical problems caused by slow oxidation in the transportation process and continuous oxidation of IPA during end use at a high temperature, i.e., the IPA treatment process, and finally can better reduce impurity residues and corresponding particle pollution caused by IPA oxidation.

In the embodiments of the present disclosure, a pipeline for supplying a mixture of nitrogen and hydrogen is added on the basis of the existing machine, and is led to the IPA tank. The mixture is charged till an oxygen-free environment is established, so as to prevent an IPA oxidation reaction; besides, hydrogen can be used to reduce a small amount of oxidized acetone, thereby the amount of residues can be greatly reduced.

The process method using IPA according to the embodiments of the present disclosure can be realized by using the process device using IPA shown in FIG. 1, and comprises the following steps:

Step S101: storing IPA in a process tank 102;

Step S102: introducing a shielding gas into the process tank 102, which provides an oxygen-free atmosphere in the process tank, thereby prevents the IPA from being oxidized;

In the method according to the embodiments of the present disclosure, the shielding gas at least comprises one or more inert gases or nitrogen, which is used to realize the oxygen-free atmosphere.

The shielding gas at least comprises one or more reducing gases, which are used to reduce IPA oxides in the process tank 102. The IPA oxide comprises acetone.

In the method according to some preferred embodiments, the reducing gas comprises hydrogen.

The shielding gas employs a mixture of oxygen and hydrogen.

Step S103: delivering the IPA to a process position and realizing an IPA treatment process. The IPA treatment process comprises an IPA drying process.

In the method according to some embodiments, the IPA further comprises heating the IPA before the IPA is delivered to the process position.

The maximum temperature in the IPA drying process is equal to or above 70 degrees.

In the method according to the embodiments of the present disclosure, by introducing the shielding gas before the IPA treatment process, IPA oxidation can be prevented in the IPA treatment process, thereby corresponding impurity residues and particle pollution caused by IPA oxidation can be prevented.

In the method according to the embodiments of the present disclosure, a mixture of nitrogen and hydrogen is introduced into the IPA tank before the process is started to ensure that the IPA is fully in an oxygen-free atmosphere during use, and oxygen is isolated to prevent the IPA from being continuously oxidized, while a small amount of oxidation product such as acetone is reduced. In the method according to the embodiments of the invention, a proper amount of a mixture of nitrogen and hydrogen is introduced into the IPA tank to prevent the oxidation reaction of IPA and reduce a small amount of oxidation products formed in the transportation chain, so that the problems of slow oxidation of IPA during transportation and continuous oxidation of IPA during end use at a high temperature can be solved.

While the present disclosure is described above in detail in some embodiments, the present disclosure is not limited to those embodiments. Various variations and modifications can be made by those skilled in the art without departing from the principles of the present disclosure, but such variations and modifications should be deemed as falling in the scope of protection of the present disclosure.

What is claimed is:

1. A process device using IPA, comprising:
a process tank for storing IPA, wherein
the process tank comprises a first inlet and a second inlet;
the first inlet is connected to an IPA supply through a first pipeline, and the IPA supply is used to supply the IPA into the process tank; and
the second inlet is connected to a shielding gas supply through a second pipeline, and the shielding gas supply is used to introduce a shielding gas into the process tank; in the process tank, the shielding gas provides an oxygen-free atmosphere, thereby prevents the IPA from being oxidized.

2. The process device using IPA according to claim 1, wherein the shielding gas at least comprises one or more inert gases or nitrogen, which is used to realize the oxygen-free atmosphere.

3. The process device using IPA according to claim 2, wherein the shielding gas at least comprises one or more reducing gases, which are used to reduce IPA oxides in the process tank.

4. The process device using IPA according to claim 3, wherein the reducing gas comprises hydrogen.

5. The process device using IPA according to claim 4, wherein the shielding gas employs a mixture of oxygen and hydrogen.

6. The process device using IPA according to claim 3, wherein the IPA oxide comprises acetone.

7. The process device using IPA according to claim 1, wherein the process tank comprises a first outlet, a second outlet and a third outlet;
the first outlet is connected to an IPA drain through a third pipeline;
the second outlet is connected to a shielding gas drain through a fourth pipeline;
the third outlet is connected to a process tower through a fifth pipeline, and the process tower is used to deliver the IPA to a process position and realize an IPA treatment process.

8. The process device using IPA according to claim 7, wherein the IPA treatment process comprises an IPA drying process.

9. The process device using IPA according to claim 8, wherein a heater is arranged on the fifth pipeline for heating the IPA flowing therethrough to a temperature required by the IPA treatment process.

10. The process device using IPA according to claim 9, wherein the maximum temperature in the IPA drying process is equal to or above 70 degrees.

11. The process device using IPA according to claim 9, wherein downstream of the heater, the fifth pipeline is connected to the third inlet of the process tank through a sixth pipeline for refluxing the IPA into the process tank.

12. A process method using IPA, comprising the following steps:
storing IPA in a process tank;
introducing a shielding gas into the process tank, which provides an oxygen-free atmosphere in the process tank, thereby prevents the IPA from being oxidized; and
delivering the IPA to a process position and realizing an IPA treatment process.

13. The process method using IPA according to claim 12, wherein the shielding gas at least comprises one or more inert gases or nitrogen, which is used to realize the oxygen-free atmosphere.

14. The process device using IPA according to claim 13, wherein the shielding gas at least comprises one or more reducing gases, which are used to reduce IPA oxides in the process tank.

15. The process device using IPA according to claim 14, wherein the reducing gas comprises hydrogen.

16. The process device using IPA according to claim 15, wherein the shielding gas employs a mixture of oxygen and hydrogen.

17. The process device using IPA according to claim 14, wherein the IPA oxide comprises acetone.

18. The process device using IPA according to claim 7, wherein the IPA treatment process comprises an IPA drying process.

19. The process device using IPA according to claim 18, wherein the IPA further comprises heating the IPA before the IPA is delivered to the process position.

20. The process device using IPA according to claim 19, wherein the maximum temperature in the IPA drying process is equal to or above 70 degrees.
